(19) United States
(12) Patent Application Publication
(10) Pub. No.: US 2015/0321028 A1
(43) Pub. Date: Nov. 12, 2015

(54) Tablet Deodorant Device

(71) Applicant: Mark Bennett, St. Johns (CA)

(72) Inventor: Mark Bennett, St. Johns (CA)

(21) Appl. No.: 14/273,000

(22) Filed: May 8, 2014

(51) Int. Cl. A61Q 15/00; B65D 17/00; B65D 75/36; B65D 77/04; A61K 8/02; B65D 75/52

(52) U.S. Cl. CPC A61Q 15/00; A61K 8/0233; B65D 75/522; B65D 75/367; B65D 77/04; B65D 17/24; A61K 2800/88; A61K 2800/87; B65D 2575/362

(57) ABSTRACT

A tablet deodorant device facilitates transport and storage of individual portions of deodorant while away from home. The device includes a tablet constructed of a deodorant material and a coating encasing the tablet. The coating has a melting point such that the coating is configured for melting upon being positioned in an armpit wherein the deodorant material is dispersed into the armpit.

### BACKGROUND OF THE DISCLOSURE

### Field of the Disclosure

The disclosure relates to deodorant devices and more particularly pertains to a new deodorant device for facilitating transport and storage of individual portions of deodorant while away from home.

### SUMMARY OF THE DISCLOSURE

An embodiment of the disclosure meets the needs presented above by generally comprising a tablet constructed of a deodorant material and a coating encasing the tablet.

The coating has a melting point such that the coating is configured for melting upon being positioned in an armpit wherein the deodorant material is dispersed into the armpit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a partial cut-away view of a tablet deodorant device according to an embodiment of the disclosure.

FIG. 2 is a partially exploded top front side perspective view of an embodiment of the disclosure packaged for storage prior to use.

FIG. 3 is a top view of an embodiment of the disclosure.

FIG. 4 is a cross-sectional view of an embodiment of the disclosure taken along line 4-4 of FIG. 1.

FIG. 5 is a top front side perspective view of an embodiment of the disclosure being applied for use.

### DESCRIPTION OF THE PREFERRED EMBODIMENT

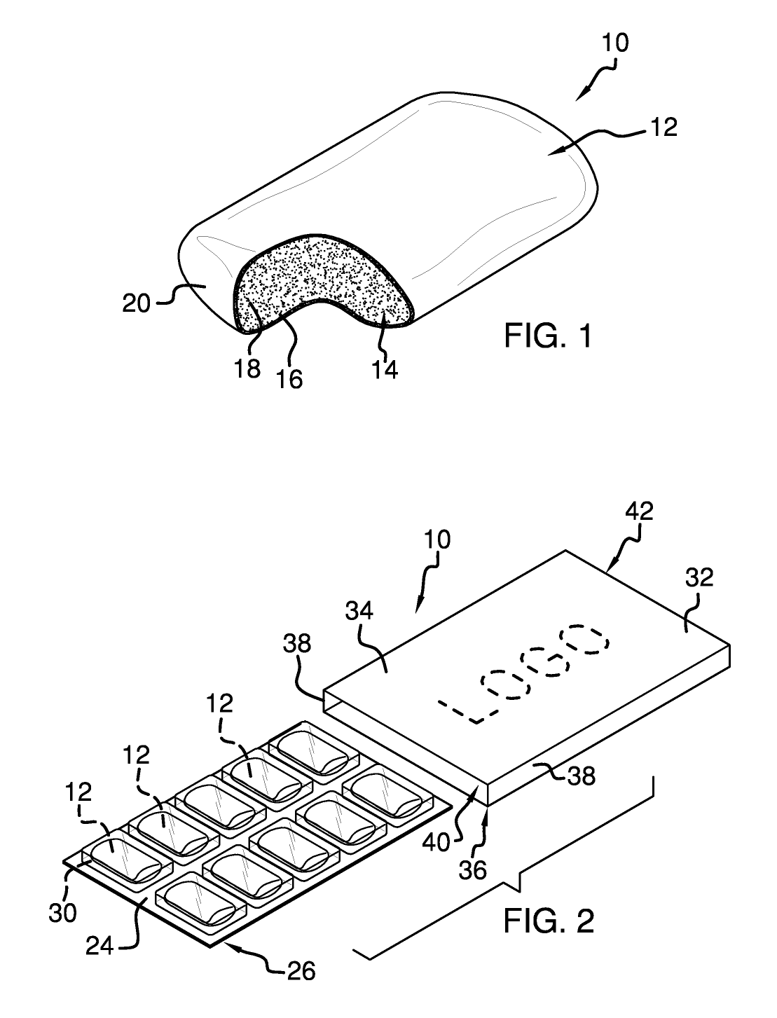

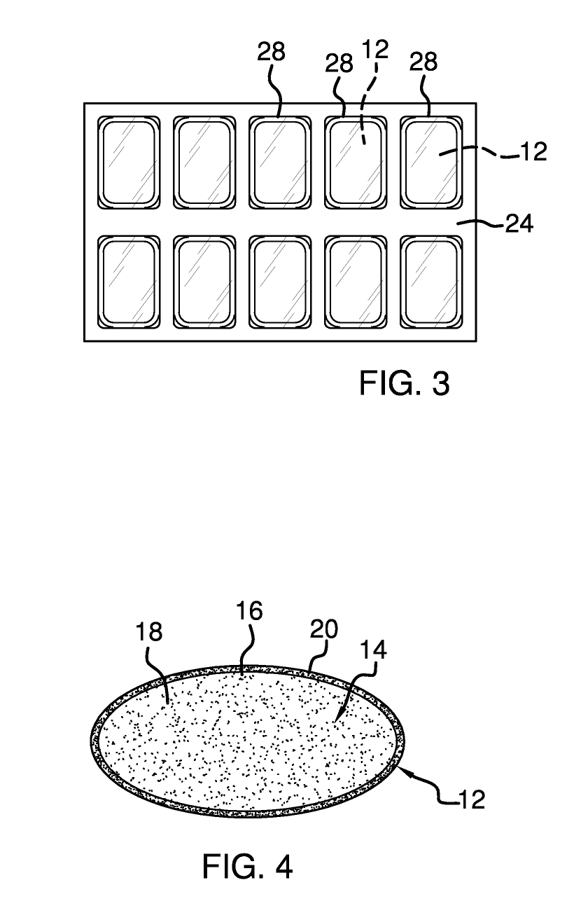

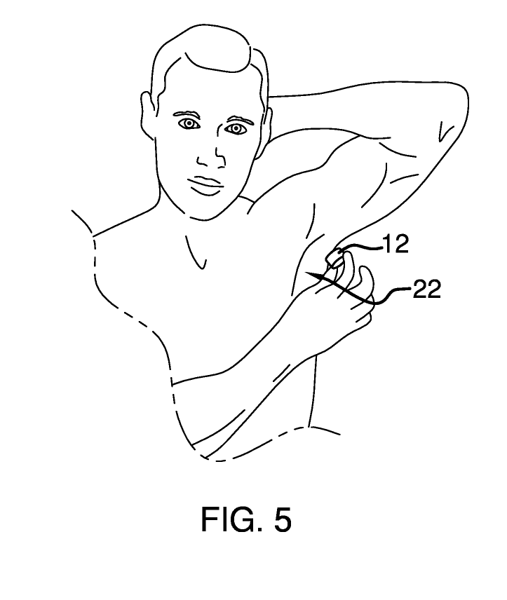

With reference now to the drawings, and in particular to FIGS. 1 through 5 thereof, a new deodorant device embodying the principles and concepts of an embodiment of the disclosure and generally designated by the reference numeral 10 will be described.

As best illustrated in FIGS. 1 through 5, the tablet deodorant device 10 generally comprises a plurality of tablets 12. Each tablet 12 is constructed of a deodorant material 16 of a type conventionally known. The deodorant material 16 may be a gel 14. Each tablet 12 may further comprise an anti-perspirant material 18 combined with the deodorant material 16 in a conventional manner used in currently available deodorant products. A respective coating 20 encases each tablet 12 preserving the tablet 12 and preventing premature dispensing of the deodorant material 16. The coating 20 is of a conventional type having a melting point such that the coating 20 is configured for melting upon being positioned in an armpit 22 wherein the deodorant material 16 is dispersed into the armpit 22. The coating 20 is further constructed of a conventional material known to dissipate and evaporate subsequent to melting such that the coating 20 leaves no substantial residual in the armpit 22.

A container 24 is provided having a back face 26 and a plurality of compartments 28. The container 24 is transparent and may be formed of a molded plastic or the like. Each of the tablets 12 is positioned in an associated one of the compartments 28 protecting the integrity of the coating 20 and the tablet 12 until ready for use. The back face 26 is planar and a backing 30 is coupled to the back face 26 of the container 24. The backing 30 covers the compartments 28. The backing 30 is constructed of a conventional plastic, foil, combination of plastic and foil, or the like to be frangible wherein the backing 30 is configured for providing access to a selectable compartment 28 through the backing 30. A cover 32 has a front face 34, a back face 36, a pair of longitudinal side faces 38, and an open end 40 wherein the container 24 is insertable into the cover 32. A second end 42 of the cover 32 may also be open or may be closed. The cover 32 is opaque wherein the cover 32 is configured for preventing the container 24 from being exposed to light while the container 24 is positioned in the cover 32. Thus, the cover inhibits heating of the coating 20 and tablet 12 in each compartment 28 prior to the intended use.

In use, each tablet 12 provides an effective amount of the deodorant material 16 for a single application. As needed, a single tablet 12 is positioned in the armpit 22 allowing the coating 20 to dissipate and dispensing the deodorant material 16 into the armpit 22. A second tablet 12 may then be applied to a second armpit 22. The container 24 may be easily transported and stored for use while traveling or otherwise being away from home, such as at a gym or the like.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A tablet deodorant device comprising:
a tablet, said tablet being constructed of a deodorant material; and
a coating encasing said tablet, said coating having a melting point such that said coating is configured for melting upon being positioned in an armpit wherein said deodorant material is dispersed into the armpit.

2. The device of claim 1, further comprising:
a plurality of said tablets, each said tablet being encased by a respective said coating; and
a container having a back face and a plurality of compartments, each of said tablets being positioned in an associated one of said compartments, said back face being planar; and
a backing coupled to said back face of said container, said backing covering said compartments, said backing being frangible wherein said backing is configured for providing access to a selectable compartment through said backing.

3. The device of claim 1, further comprising said tablet comprising an anti-perspirant combined with said deodorant material.

4. The device of claim 2, further comprising a cover, said cover having a front face, a back face, a pair of longitudinal side faces, and an open end wherein said container is insertable into said cover.

5. The device of claim 4, further comprising said container being transparent.

6. The device of claim 5, further comprising said cover being opaque wherein said cover is configured for preventing said container from being exposed to light while said container is positioned in said cover.

7. The device of claim 1, further comprising said deodorant material being a gel.

8. A tablet deodorant device comprising:
a plurality of tablets, each said tablet being constructed of a deodorant material, said deodorant material being a gel, each said tablet comprising an anti-perspirant combined with said deodorant material;
a respective coating encasing each said tablet, said coating having a melting point such that said coating is configured for melting upon being positioned in an armpit wherein said deodorant material is dispersed into the armpit;
a container having a back face and a plurality of compartments, said container being transparent, each of said tablets being positioned in an associated one of said compartments, said back face being planar; and
a backing coupled to said back face of said container, said backing covering said compartments, said backing being frangible wherein said backing is configured for providing access to a selectable compartment through said backing; and
a cover, said cover having a front face, a back face, a pair of longitudinal side faces, and an open end wherein said container is insertable into said cover, said cover being opaque wherein said cover is configured for preventing said container from being exposed to light while said container is positioned in said cover.
